(19) United States
(12) Patent Application Publication
(10) Pub. No.: US 2007/0027425 A1
(43) Pub. Date: Feb. 1, 2007

(54) Collapsible tampon applicator

(75) Inventors: Thomas Ward Osborn III, Clifton, OH (US); Diana Lynne Gann, Lebanon, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/190,658

(22) Filed: Jul. 27, 2005

(51) Int. Cl. A61F 13/20

(52) U.S. Cl. 604/16

(57) ABSTRACT

A tampon applicator for positioning a tampon inside a vaginal cavity. The tampon applicator includes an outer member for housing a tampon. The outer member has a substantially rigid first section and a collapsible section adjacent to the first section. The tampon positioning member is disposed within the outer member and co-axially with the tampon for expelling the tampon from the outer member and for placing the tampon into the proper position within the vaginal cavity.

### FIELD OF INVENTION

This invention relates to a tampon applicator, particularly, to a collapsible tampon applicator providing proper placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and houses a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal and surrounding organs, further reducing the tampon coverage of menses flow in the vaginal cavity. Moreover, current tampon applicators are designed to “push” the tampon out of the outer tube substantially higher than the tip of the outer tube. This often causes the tampon to be deflected by the cervix/anterior fornix, resulting in an off-centered position of the tampon, and, thus, result in possible leakage from the vaginal cavity.

Alternatively, tampon applicators can place a tampon too low in the vaginal cavity. When the tampon is placed too low in the vaginal cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, it would be beneficial to provide a tampon applicator which properly places the tampon at a desired position in the vaginal cavity.

### SUMMARY OF THE INVENTION

The present invention encompasses a tampon applicator for positioning a tampon into a proper position inside a vaginal cavity. The tampon applicator has a tampon which is partially housed within an outer member. The outer member has a substantially rigid first section and a collapsible section adjacent to the first section. The collapsible section can comprise plastic material comprising a multiplicity of corrugations. The tampon positioning member is disposed within the outer member and co-axially with the tampon for expelling the tampon from the outer member and for placing the tampon into the proper position within the vaginal cavity. The tampon positioning member could comprise a projection extending radially from within the outer member or a tubular member. The tampon remains in contact with the tampon positioning member at the most proximal position of the tampon applicator.

Alternatively, the tampon applicator can further comprise a substantially rigid second section abutting the collapsible section. In one non-limiting example, the tampon positioning member is fixedly joined to the outer member. Alternatively, the tampon positioning member can be slideably joined to the outer member.

The tampon applicator has a collapsed length and the tampon is at least partially housed within the outer member while the tampon applicator is in the collapsed length. The collapsible section collapses at a force of less than about 0.3 kg, more preferably less than about 0.15 kg. The collapsible section comprises an initial length. The initial length collapses to less than about 60% of the initial length.

All documents cited are, in relevant part, incorporated herein by reference; the citation of any document is not to be construed as an admission that it is prior art with respect to the present invention.

### BRIEF DESCRIPTION OF THE DRAWINGS

FIG. 1 is a perspective view of a tampon applicator of the present invention with a tampon in its expanded state.

FIG. 2 is a perspective view of the tampon applicator while the tampon applicator has a collapsed length.

FIG. 3 is a perspective view of an alternative embodiment of the tampon applicator of the present invention with the tampon.

FIG. 4 is a perspective view of another alternative embodiment of the tampon applicator of the present invention with the tampon.

FIG. 5 is a perspective view of another alternative embodiment of the tampon applicator of the present invention with the tampon.

### DETAILED DESCRIPTION OF THE INVENTION

Section A will provide terms which will assist the reader in best understanding the features of the invention, but not to introduce limitations in the terms inconsistent with the context in which they are used in this specification. These definitions are not intended to be limiting. Section B will discuss the tampon applicator of the present invention.

A. Terms

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 mm long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 mm wide corresponding to the largest cylindrical cross section. The width can vary along the length of the self-sustaining tampon.

The second type of tampon is an easily “deformable, fluid-permeable bag tampon”. The deformable, fluid-permeable bag tampon consists of absorbent chips, spheres, or fibers such that the fluid permeable bag tampon is readily deformable with a force of less than about 3 psi. The tampon is substantially deformable at pressures of less than about 1 psi; resulting in the tampon spreading or being easily indented when the pressure is applied from a surface of about 0.15 mm diameter.

As used herein, the terms “vaginal cavity,” “within the vagina,” and “vaginal canal” refer to the internal genitalia of the human female in the pudendal region of the body. The term “vaginal canal” is not intended to include the interlabial space including the floor of the vestibule. The externally visible genitalia generally are not included within the term “vaginal canal” as used herein.

As used herein, the term “hymen ring” refers to the demarcation between the vaginal cavity and external genitalia and is identified by the position of the hymen or residual tissue of the hymen.

As used herein, the term “proper position” refers to a vertical position of the tampon inside the vaginal cavity, wherein the base of the tampon (i.e., the main absorbent material) is positioned at least about 5 mm above the hymen ring and more preferably about 10 mm above the hymen ring. The base of the tampon does not include any overwrap, secondary absorbent member, or withdrawal cord which extends beyond the tampon's main absorbent material.

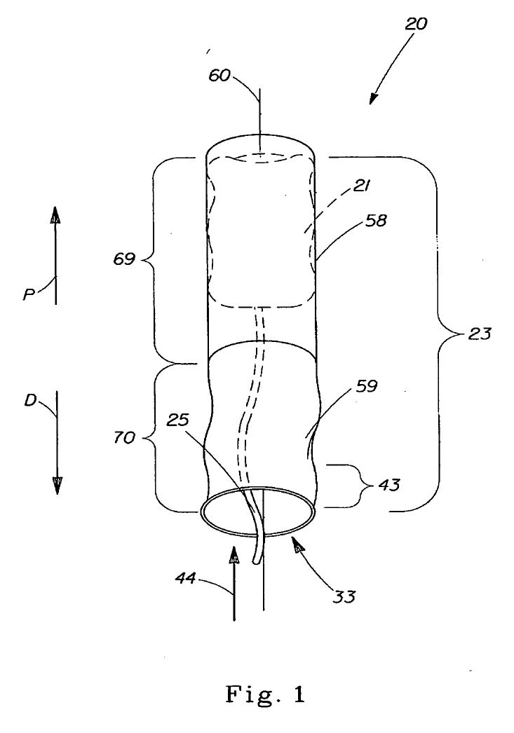

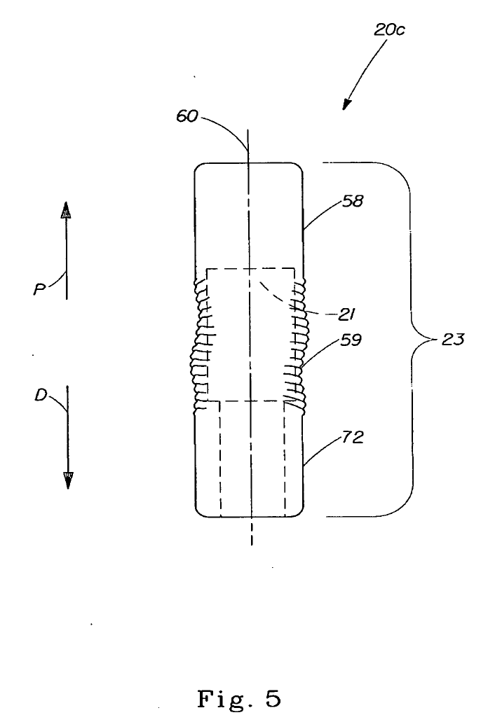

The terms “distal” and “proximal,” respectively designated P and D in FIG. 1 and FIG. 5, herein respectively refer to directions away from and towards the body of the tampon wearer unless otherwise specified.

As used herein, the term “expand” means that the tampon applicator can be enlarged to become greater in size, bulk, or volume.

By the term “expanded length,” it is meant herein the length of the tampon applicator when the most proximal end and the most distal end of the tampon applicator is stretched out.

The term “collapsed length” refers to the fully collapsed length of the tampon applicator during expulsion of the tampon from the tampon applicator. While the tampon applicator has a collapsed length, the tampon can still be partially housed in the tampon applicator.

The term “fully expelled” refers to as the base of the tampon disengaged from the all portions of the tampon applicator when the tampon positioning member is at the proximal position. The base of the tampon does not include any overwrap, secondary absorbent member, or withdrawal cord which extends beyond the tampon's main absorbent material.

The term “partially expelled,” as used herein, is meant that the base of the tampon partially retained within a portion of the applicator when the tampon positioning member is at a proximal position. The base of the tampon does not include any overwrap, secondary absorbent member, or withdrawal cord which extends beyond the tampon's main absorbent material.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Tampon Applicator of the Present Invention

Referring to FIG. 1, a tampon applicator 20 is shown which is designed to position tampon 21 to achieve a proper position inside the vaginal cavity. Also, FIG. 1 shows the “proximal” and “distal” designated as P and D, respectively. The tampon applicator 20 can be used with any type of tampon 21. The tampon 21 could be a self-sustaining tampon or a deformable, fluid-permeable bag tampon. Generally, the tampon applicator 20 includes an outer member 23 and a tampon positioning member 25.

The outer member 23 houses at least a portion of the tampon 21. The outer member 23 comprises a hollow interior 33, a first end 69, and a second end 70 opposed to the first end 69. The first end 69 is the most proximal end of the outer member 23 along a longitudinal axis 60. In addition, the first end 69 is the portion of the outer member 23 in which the tampon applicator 20 can be inserted into the vaginal cavity and it is the end from which the tampon 21 can be expelled. The second end 70 is the most distal end of the outer member 23 along the longitudinal axis 60. In addition, the second end 70 can be the portion of the outer member 23 in which the actuating force 44 is applied along the longitudinal axis 60 to expel the tampon 21, as well as the gripping portion of the outer member 23 during insertion into the vaginal cavity.

The first end 69 of the tampon applicator 20 can comprise a collapsible section 59 and/or a first rigid section 58. Alternatively, the second end 70 of the tampon applicator 20 can comprise a collapsible section 59 and/or a first rigid section 58. The application of force 44 applied to the collapsible section 59 results in a change of length of the tampon applicator 20 during insertion of the tampon applicator 20 into the vaginal cavity to allow the tampon 21 to be properly placed in the vaginal cavity. Specifically, during insertion, the collapsible section 59 allows a change in the length of the tampon applicator 20 from an expanded length to a collapsible length. The first rigid section 58 serves to provide stability for the tampon applicator 20 during insertion of the tampon applicator 20 into the vaginal cavity.

In addition to the tampon applicator 20 comprising an outer member 23, the tampon applicator 20 comprises the tampon positioning member 25. The tampon positioning member 25 positions the tampon 21 along the length of the outer member 23 so that during expulsion of the tampon 21 from the tampon applicator 20 the tampon 21 will travel a predetermined distance. The tampon positioning member 25 can be slidably joined to the outer member 23.

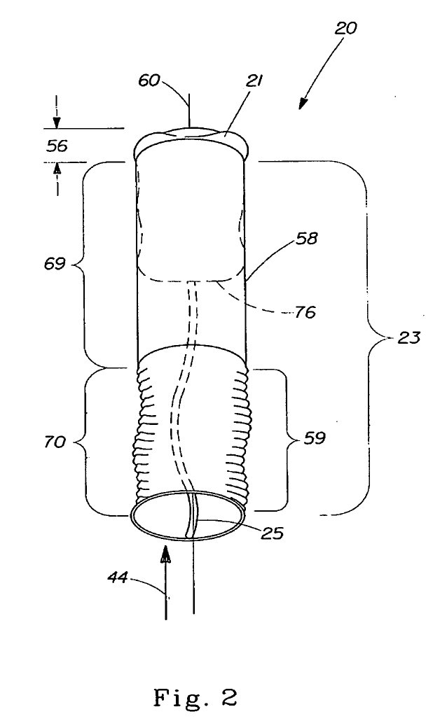

To use the tampon applicator 20 of the present invention, the user can hold the grip region 43 located in the second end 70 with her thumb and index finger. While holding the grip region 43, the user can insert the tampon applicator 20 into the vaginal cavity with a force 44 applied along the longitudinal axis 60. As shown in FIG. 2, while the force 44 is applied along the longitudinal axis 60, the tampon positioning member 25 slides toward the first end 69 of the outer member 23 and can bear against the tampon abutting portion 76 leaving the exposed portion 56 of the tampon 21 revealed within the vaginal cavity. In addition, while the force 44 is applied to the tampon applicator 20, the collapsible section 59, which can be located in the second end 70, collapses, and the first rigid section 58 remains rigid as compared to the collapsible section 59. During removal of the tampon applicator 20 from the body cavity, the body tissues hold onto the tampon 21. Because the body tissues hold onto the tampon 21, the tampon applicator 20 exits the body cavity without the tampon 21, i.e., leaving the tampon 21 behind. This results in proper placement of the tampon 21 within the vaginal cavity.

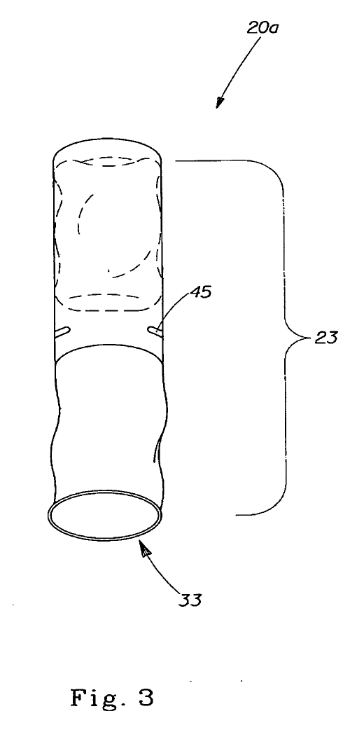

Referring to FIG. 3, in an alternative embodiment of the tampon applicator 20A, the tampon positioning member can be an annular ring 45 adjacent to the interior 33 of the outer member 23.

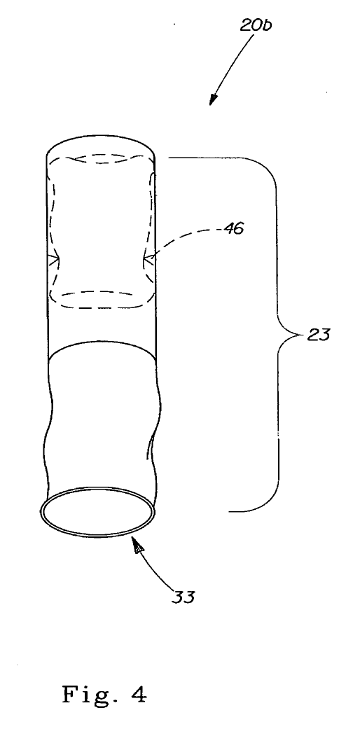

Referring to FIG. 4, in another alternative embodiment of the tampon applicator 20B, the tampon positioning member can be a protuberance 46 adjacent to the interior 33 of the outer member 23.

Referring to FIG. 5, in another alternative embodiment of the tampon applicator 20C with the tampon 21 of the present invention is shown, showing the terms “proximal” and “distal” designated as P and D, respectively. In this alternative embodiment, the first rigid section 58 can be located at the most proximal end of the outer member 23 along the longitudinal axis 60, a second rigid section 72 can be located at the most distal end of the outer member 23 along the longitudinal axis 60, and the collapsible section 59 can be located between the first rigid section 58 and the second rigid section 72.

In addition to delivering menstrual tampons into the vaginal cavity, it should be noted that the tampon applicator of the present invention can be used to deliver any other type of absorbent or nonabsorbent object to any suitable cavity. For example, the tampon applicator of the present invention could be used to insert incontinence inserts. An “incontinence insert,” as used herein refers to devices specifically designed, configured, and/or adapted for placement into a vagina in order to reduce the occurrence and/or severity of female urinary incontinence. While incontinence inserts are typically made of non-absorbent materials, at least partially absorbent materials may also be used. However, because there is no intent to absorb bodily fluids, and because the incontinence inserts are adapted and configured to provide structural support to the musculature and body tissues located near the urethra-vaginal myfascial area, incontinence inserts are readily distinguishable from catamenial tampons.

Below will detail each component of the tampon applicator of the present invention.

i Outer Member

Referring to FIG. 1, typically, the outer member 23 can be used to handle or grip the tampon applicator 20 during insertion into the vaginal cavity. The outer member 23 has a hollow interior 33, a first end 69, and the second end 70 opposed to the first end 69. The first end 69 is the most proximal end of the outer member 23 along the longitudinal axis 60. In addition, the first end 69 can be inserted into the vaginal cavity and it is the end from which the tampon 21 can be expelled. The second end 70 is the most distal end of the outer member 23 along the longitudinal axis 60. In addition, the second end 70 can be the portion of the outer member 23 in which the actuating force 44 is applied along the longitudinal axis 60 to expel the tampon 21 and can be the gripping portion of the outer member 23 during insertion into the vaginal cavity.

The manufacturer of tampon applicator 20 can vary the size of the outer member 23. The size of the outer member 23 can be determined primarily by the dimensions of tampon 21. The diameter of the outer member 23 can be varied to accommodate different absorbency tampons. Generally, the outer member 23 can have a diameter of from about 8 millimeters to about 21 millimeters and a thickness of from about 0.4 millimeter to about 1.2 millimeter. In addition, the diameter of the outer member 23 can be at least from about 1 millimeter to about 2 millimeters greater than the diameter of the tampon 21 which prevents the outer member 23 from interfering with the removal of the tampon 21 from the outer member 23. Moreover, the outer member 23 can have varying diameters to conform to the profiled shape of the enclosed tampon 21.

Also, the manufacturer of tampon applicator 20 can vary the length of the outer member 23. The outer member 23 should be of a sufficient length to house or contain at least a portion of the tampon 21 prior to expulsion of the tampon 21 from the tampon applicator 20 into the vaginal cavity. Generally, the length of the outer member 23 can be from about 50 millimeters to about 85 millimeters. In one non-limiting example, the length of the outer member 23 can be about 50 millimeters. In yet another non-limiting example, the length of the outer member 23 can be about 85 millimeters. The length of the outer member 23 is preferably from about 50 mm to about 100 mm, more preferably from about 55 mm to about 85 mm. More specific examples are described below.

The outer member 23 is in no way limited by the shape that it can assume except that the shape should not hinder directional expulsion of the tampon 21. It is further noted herein that the shape of the outer member 23 can vary as long as a portion of the tampon positioning member 25 can be adjacent, internal, joined, integral, and/or monolithic (by molding the tampon positioning member 25 and the outer member 23 as one piece) with the outer member 23. One skilled in the art can imagine other shapes of the outer member 23, for example, cylindrical or curved like a banana or any other suitable shape possible as long as any such shape would work effectively to properly aid in the expulsion of the exposed portion 56 of the tampon 21 (shown in FIG. 2) from the tampon applicator 20 and comfortably insert into the vaginal cavity. The outer member 23 can be of any suitable cross-sectional shape including, but not limited to, circular, oval, flattened, circular, elliptical, and any combination thereof.

The outer member 23 can comprise the grip region 43 having a perimeter. Specifically, the second end 70 of the outer member 23 can contain the grip region 43. The grip region 43 can provide for secure handling of the outer member 23. The perimeter of the grip region 43 can take essentially any desired shape, including oval, circular, and various other geometric patterns. The gripping region 43 can be created by outward projections, raised surfaces, or by impressing or compressing the surfaces. Referring again to FIG. 1, the grip region 43 is substantially circularly shaped, but can also take on more angular formations such as squared. The surface of the grip region 43 can be any kind of surface known in the art. This surface area can provide a desired frictional resistance during insertion of the tampon applicator 20 into the body. Moreover, this surface area can have, for example, a dove pattern created via surface texturing, impressions, and/or indentations. As shown in FIG. 5, the grip region 43 can be convex to assist the user in achieving a secure grip.

Furthermore, it is preferable that the grip region 43 be of sufficient dimension to substantially accept a user's grip. As used in the specification and claims, the term “user's grip” means any way of holding the tampon applicator 20 in a hand, e.g., between a thumb and a finger. The grip region 43 need not necessarily extend completely around the perimeter of the outer member 23. For example, a space can be provided for a decorative marking or a Trademark character.

The outer member 23 has the collapsible section 59 and the first rigid section 58. The first rigid section 58 may be adjacent to the collapsible section 59. The collapsible section 59 allows the user to change the configuration during insertion of the tampon applicator 20 into the vaginal cavity to allow the tampon 21 to be properly placed in the vaginal cavity. Specifically, during insertion, the collapsible section 59 allows the tampon applicator 20 to change from an expanded length to a collapsible length. Typically, the collapsible section 59 is shorter in length along the longitudinal axis 60 than the expanded length. The collapsible section 59 can be alternately collapsible and extensible. The first rigid section 58 can be designed to provide more stability during the insertion of the tampon applicator 20. The first rigid section 58 is substantially rigid, at least as compared to the collapsible section 59.

At the same time, additional rigid sections and additional collapsible sections can be added as long as there is at least one collapsible section 59 and at least one rigid section 58 included in the outer member 23. For example, referring to FIG. 5, a second rigid section 72 can be added to the outer member 23 of tampon applicator 20C. In this non-limiting example, the first rigid section 58 can be located at the most proximal end of the outer member 23 along the longitudinal axis 60, the second rigid section 72 can be located at the most distal end of the outer member 23 along the longitudinal axis 60, and the collapsible section 59 can be located between the first rigid section 58 and the second rigid section 72. In this example, moreover, the collapsible section 59 can be alternately collapsible and extensible, while the first and second rigid sections 58 and 72 are substantially rigid, at least as compared to the collapsible section 59. When the second rigid section 72 is added, the bottom of the tampon positioning member 25 can be mounted at the most distal end of the second rigid section 72. Additionally, in this non-limiting example, the user can grasp the second rigid section 72.

The rigid section(s) and/or the collapsible section(s) can be configured in any way as long as the tampon applicator 20 partially expels the tampon 21 from the tampon applicator. For example, the outer member 23 could comprise the following sequence beginning with the most proximal end of the tampon applicator and ending with the most distal end of the tampon applicator along the longitudinal axis 60: a first collapsible section, a first rigid section, a second collapsible section, a second rigid section, and a third collapsible section. In another example, the outer member 23 can comprise the following sequence beginning with the most proximal end of the tampon applicator and ending with the most distal end of the tampon applicator along the longitudinal axis 60: a first rigid section, a second rigid section, and a collapsible section. Thus, two rigid sections can be adjacent to one another. Likewise, two collapsible sections can be adjacent to one another.

The rigid section(s) and/or the collapsible section(s) can be any convenient size or shape. Moreover, the rigid section(s) and/or the collapsible section(s) can encompass the entire outer member 23 or may encompass part of the outer member 23. The size of the rigid section(s) and/or the collapsible section(s) may encompass any design. Also, the design can be any shape such as rectangular, square, elliptical, polygonal, triangular, etc. The design can encompass one shape or a combination of the afore-mentioned shapes. Additionally, the design can encompass one color or several colors.

The force 44 applied along the longitudinal axis 60 required for the collapsible section 59 to begin collapsing during insertion of the tampon applicator of the present invention should be less than about 0.3 kg. Any material can be used for the collapsible section 59 as long as the force 44 used to collapse the collapsible section 59 is less than about 0.3 kg, more preferably about. In one non-limiting example, the collapsible section 59 can be made from low density polyethylene, and the force 44 that will be required to collapse the collapsible section 59 can be about 0.07 kg.

The maximum collapsibility of the collapsible section 59, when a force 44 is applied along the longitudinal axis 60, can be at least about 20% and no more than about 99% of the starting length of the collapsible section 59. Any material can be used for the collapsible section 59 as long as the maximum collapsibility of the collapsible section 59 can be at least about 20% and no more than about 99% of the starting length of the collapsible section 59. In one non-limiting example, the collapsible section 59 is made from low density polyethylene and the starting length is about 35 millimeters, the collapsible section 59 collapses to a maximum of about 34 millimeters. In another non-limiting example, the collapsible section 59 is made from low density polyethylene and the starting length is about 35 millimeters, the collapsible section 59 collapses to a maximum of about 26 millimeters.

The collapsible section 59 can be configured or shaped in any manner as long as the collapsible section 59 collapses partially or fully when the actuating force 44 is applied to the tampon positioning member 25 which in turn pushes the tampon 21 toward the first end 69 of the tampon applicator 20. The diameter of the collapsible section 59 can be from about 1 millimeter to about 4 millimeters greater than the diameter of the tampon 21 to prevent the outer member 23 from interfering with the expulsion of the tampon 21 from the outer member 23.

The length of the collapsible section 59 can be any length as long as the tampon 21 can be partially expelled when the collapsible section 59 is fully collapsed. In one non-limiting example, the length of the collapsible section can be from about 20 millimeters to about 36 millimeters when the length of the tampon 21 is about 48 millimeters.

a. Material of the Collapsible Portion

The collapsible section 59 can be any material which is bendable, flexible, and collapses during insertion of the tampon applicator of the present invention with a force of less than about 0.3 kg applied to the tampon applicator 20. Typically, the collapsible section 59 can be more collapsible when the material is thinner. The collapsible section 59 can comprise perforations such as slits, slots, cut-outs, and the like (including perforations which extend through less than the complete thickness of the material of the collapsible section 59 or one or more layers of the material of the collapsible section 59); folds; scores; and any combination thereof. The collapsible section 59 can also comprise a material that is inherently flexible, such as latex or rubber.

Moreover, the collapsible section 59 may be made of any type of planar, flexible film, or other deformable substrate. The typical film material used to make a collapsible section 59 may be a sheet-shaped substrate that may be smooth or embossed. Some materials include paper, woven and non-woven substrates, cloths, metal foils, cellulose fibre sheets, and organic polymeric materials.

Film materials may be single layer, planar laminate, or comprise of connected structures of two or more film materials joined to each other in a horizontal side-by side arrangement, for example, lap or edge-to-edge butt joints to form the film. Such joined materials may leave a weld or seam, though care should be taken to minimize the length, width, and height of such welds. Generally, the welds may be flattened out or covered by a coating, prior to use of the joined film to make the collapsible section 59. In typical embodiments of the invention, the film used to make the collapsible section 59 has no welds or seams.

Films may be made of one or more of the following substrates and resins: polyolefins, cellulose materials and derivatives, including cellulose ethers, ethyl and/or methyl celluloses; cellulose esters, including cellulose acetates and/or formats; vinyl polymer derivatives or more typically cellophane and/or polyethylenes; polypropylenes; latex; nylon; polyesters; and polystyrenes. Some other resins and films include polylactides; polyester amides; aliphatic esters; aliphatic-aromatic copolyesters; polyhydroxyalkonoates; polyalkulene succinate; polyvinyl alcohols, cellulosic polymers; starch-based materials; and/or polycaprolactone. Polyolefin, such as polyethylenes and polypropylenes and/or biodegradable films, are typical. Polyolefins are also described and defined in “Plastics and Films,” chapter 2, by J H. Briston, 3^{e} edition, published in 1988, Langman Scientific & Technical.

Some film and resin examples are: BAK 1095 (available from Bayer), Eastar Bio (available from Eastman Chemicals),for example, a blown 37 micron Eastar Bio film, Mater-Bi (available from Novamont), Biomax (available from DuPont), Bionelle (available from Showa High Polymer), Lunare SE (available from Nippon Shokubai), EcoPLA (available from Dow Cargill), Exoflex (available from BASF), Biotec (available from Kashoggi), Vinex (available from Air Products), cellophane (available from UCB films including the low stretchable or low yieldable uncoated cellophane of about 24.3 microns thick), LACEA (available from Mitsui), and high density polyethylenes (“HDPE”) film available from Tredegar.

Typical olefin films include in particular linear low density polyethylenes (“LLDPE”) and low density polyethylenes (“LDPE”) as available from Clopay and HDPE as available from Tredegar, including for example LDPE film of 25 microns nominal thickness, called DH215 available from Clopay.

The collapsible section 59 may be porous, micro-porous, or non-porous. The collapsible section 59 may be gas and/or water permeable. The collapsible section 59 may have a low or high critical surface tension. The collapsible section 59 may be hydrophobic or hydrophilic; thermoplastic and/or thermosetting; water soluble; water-disintegratable; or water-dispersible. The collapsible section 59 may shrink upon exposure to heat or application of pressure or vacuum. Some films that exhibit these qualities are single or biaxially oriented films, such as polypropylenes.

The film used to make the collapsible section 59 may be made by any process known in the art including casting, extrusion, or blown extrusion processes. The collapsible section 59 may have a functional coating on one or each surface that may change the film's properties such as hydrophilicity, hydrophobicity, coefficient of friction, heat sealing properties, gas and/or water permeability, colour, tactile feel and/or odour.

The film typically may have an initial thickness that may be homogenous or varied prior to formation of the collapsible section 59 and is between about 1 and about 200 microns. The thickness may be from about 5 to about 100 microns, about 10 to about 75 microns, about 15 to about 50 microns, or most typically from 20 to 40 microns. This film thickness is the calliper, measured as set out herein.

Variations in film thickness, can be created by a multitude of techniques including use of embossing the film during manufacturing, applying a coat extrusion of varying thickness to a base film, or creating a film from two or more substrates of different thickness or joining together films. Thick films such as films from about 0.1 mm to about 0.5 mm thick may be corrugated to facilitate collapse. The thicker collapsed sections may be molded during the forming process as part of the tampon applicator. The variation of film thickness from one area to the next may be large or small. The areas of weakness may be located in at least partially or only on the top portion of the collapsible section 59. For example, the areas of varied thickness are typically less than about 5%, about 3%, about 1% or even less than about 0.5% of the total surface of the collapsible section 59.

Comparing the thickness of part of the collapsible section 59 to the original thickness of the film, there is typically at least an area of the collapsible section 59 where the thickness is at least 10% less than the original film thickness. In measuring the calliper in the collapsible section 59, the thickness variation from one area to a second area may be at least about 10%, about 20%, about 40%, about 60% or even at least about 80%. Generally, variations in thickness may be at least about 20% from one area to a second area.

The variation in the thickness of the collapsible section 59 can be measured by any suitable known method. For large areas, a calliper gauge may be suitable, or method as described in U.S. Pat. No. 6,231,556. For smaller areas, a more microscopic technique is required, for example, embedding the collapsible section 59 in a setting resin, making thin cross section slices of this embedded collapsible section 59, and measuring the thickness or gauge of the collapsible section 59 in this cross section by use of, for example, a scanning electron microscope (“SEM”). The film may have breaches that have no thickness and single or multiple breaches. Breaches may include holes, perforations, slits, gaps, voids, openings, punctures, cracks, apertures, pores, etc.

b. Material of the Rigid Section(s)

The rigid sections 58 and 72 can be formed of paper, cardboard, plastic, or any combination of both. The rigid sections 58 and 72 can be formed of a spirally wound, convolutedly wound, or longitudinally seamed hollow tube that is formed from paper, paperboard, blown, cardboard, or a combination thereof. Also, the rigid sections 58 and 72 can be injection molded, extruded, or formed from flexible plastic, such as thermoformed from plastic sheet or folded or wound from plastic film. Different rigid sections 58 and 72 parts can be constructed from different materials and processes.

The rigid sections 58 and 72 can be constructed from a single ply of material or can be formed from two or more plies that are bonded together to form a laminate. The use of two or more plies or layers is preferred for it enables the manufacturer to use certain materials in the various layers that can enhance the performance of the rigid sections 58 and 72. When two or more plies are utilized, all the plies can be spirally wound, convolutedly wound, or longitudinally seamed to form an elongated cylinder. The rigid sections 58 and 72 can be constructed using a smooth thin ply of material on the outside or exterior surface that surrounds a coarser and possibly thicker inner ply. When the rigid sections 58 and 72 comprise at least three plies, the middle ply can be the thicker ply and the interior and exterior plies can be smooth and/or slippery to facilitate expulsion of the tampon 21 and to facilitate insertion of the tampon applicator 20 into the vaginal cavity. The rigid sections 58 and 72 should contain one to four plies, although more plies can be utilized.

The plies forming the rigid sections 58 and 72 can be held together by an adhesive, such as glue, heat, pressure, ultrasonic, etc. The adhesive can be either water-soluble or water-insoluble. A water-soluble adhesive is preferred for environmental reasons in that the rigid sections 58 and 72 will quickly break apart when it is immersed in water. Such immersion will occur should the rigid sections 58 and 72 be disposed of by flushing it down a toilet. Exposure of the rigid sections 58 and 72 to a municipal waste treatment plant wherein soaking in water, interaction with chemicals and agitation all occur, will cause the rigid sections 58 and 72 to break apart and evenly disperse in a relatively short period of time.

ii. Tampon Positioning Member

Referring to FIG. 2, the tampon positioning member 25 positions the tampon 21 along the length of the outer member 23 so that during expulsion the tampon 21 will travel a predetermined distance. This predetermined distance ultimately results in the tampon 21 being properly positioned in the vaginal cavity. Moreover, this position allows sufficient expulsion such that when the exposed portion 56 of the tampon 21 is expelled at least a portion of the tampon 21 is retained within the vaginal cavity by body forces.

The tampon positioning member 25 can be coaxial with tampon 21. Moreover, the tampon positioning member 25 can be fixedly joined to the outer member 23 in any way known in the art. The tampon positioning member 25 can also be monolithic with the outer member 23, for example, by molding the tampon positioning member 25 and the outer member 23 as one piece. In one non-limiting example, the tampon positioning member 25 can be fixedly joined to the outer member 23 by friction fitting that snaps parts together, gluing, and/or melting. In another non-limiting example, the tampon positioning member 25 can also be a continuation of the outer member 23 by folding or compressing a portion of the outer member 23 and turning the outer member 23 inwards upon itself to create a tube inside a tube.

During expulsion, at least a portion of the tampon 21 is adjacent to the tampon positioning member 25. In other words, when the tampon applicator 20 is at its most proximal position, the tampon positioning member 25 is in contact with the tampon 21. However, the tampon 21 may or may not be in contact with the tampon positioning member 25 before expulsion of the tampon 21 from the tampon applicator 20. Referring to FIG. 1, in one non-limiting example, before expulsion of the tampon 21 from the tampon applicator 20, the tampon positioning member 25 can be located in the hollow interior 33 of the outer member 23 without any contact with the tampon 21.

It is further noted herein that the shape or configuration of the tampon positioning member 25 can vary as long as the tampon positioning member 25 aids in positioning the tampon 21 along the length of the outer member 23 to provide proper placement of the tampon 21 within the vaginal cavity. In one embodiment, the tampon positioning member includes an elongated tampon positioning member of a generally tubular shape. Alternatively, the tampon positioning member can be a rod. The tampon abutting portion 76 can rest on top of the rod. In another embodiment, the tampon abutting portion 76 can abut the tampon positioning member 25 along the sides of the tampon 21. In another embodiment, the tampon positioning member 25 can be slideable within the outer member 23. Referring to FIG. 3, in another alternative embodiment of the tampon applicator 20A, the tampon positioning member can be an annular ring 45 adjacent to the interior 33 of the outer member 23. Alternatively, referring to FIG. 4, the tampon positioning member can be protuberances 46 adjacent to the interior 33 of the outer member 23 and extending inward from the hollow interior 33 of the outer member 23. The protuberances 46 can be of any shape or size as long as the protuberances 46 allow the tampon 21 to be in contact with the protuberances 46 during expulsion. The protuberances 46 can be fixedly joined to the outer member 23 in any way known in the art.

It is further noted herein that the length of the tampon positioning member 25 can vary as long as the tampon positioning member 25 aids the tampon 21 to be located in a position within the outer member 23 that will provide proper placement within the vaginal cavity. In one non-limiting example, the length of the tampon positioning member 25 can be from about 36 millimeters to about 63 millimeters in length.

The tampon positioning member 25 can be made of any material as long as the material allows the tampon 21 to be properly positioned along the length of the outer member 23 so that during insertion the tampon 21 will travel a predetermined distance. The materials for the tampon positioning member 25 can be made of the same materials or of different materials from the materials noted above for the rigid sections 58 and 72. Moreover, the tampon positioning member 25 can be constructed from similar materials disclosed in U.S. Pat. No. 5,346,468 issued to Campion, et al. on Sep. 13, 1994 and U.S. Pat. No. 5,558,631 issued to Campion, et al. on Sep. 24, 1996. In an alternative embodiment, the tampon positioning member 25 contains two layers. The first layer can be made of the same or different material than the second layer. The first layer may be in contact with at least a portion of the second layer.

### EXAMPLES

The following is a listing of examples illustrating various embodiments of the present invention. It would be obvious to those skilled in the art that various other changes and modifications can be made without departing from the spirit and scope of the invention.

### Example 1

The tampon applicator of the present invention can comprise the outer member of the length of 75 millimeters, the most proximal end of the tampon positioning member is located 45 millimeters from the most proximal portion of the first end of the outer member, and a tampon of the length of 30 millimeters.

### Example 2

The tampon applicator of the present invention can comprise the outer member of the length of 78 millimeters, a tampon positioning member of the length of 45 millimeters, and a tampon of the length of 33 millimeters. The outer member can have a collapsible section and a first rigid section. The length of the collapsible section is 25 millimeters. The length of the first rigid section is 53 millimeters.

### Example 3

The tampon applicator of the present invention can comprise the outer member of the length of 85 millimeters, a tampon positioning member of the length of 45 millimeters, and a tampon of the length of 40 millimeters. The outer member can have a collapsible section and a first rigid section. The length of the collapsible section is 25 millimeters. The length of the first rigid section is 60 millimeters.

### Example 4

The tampon applicator of the present invention can comprise the outer member of the length of 89 millimeters, a tampon positioning member of the length of 45 millimeters, and a tampon of the length of 44 millimeters. The outer member can have a collapsible section and a first rigid section. The length of the collapsible section is 30 millimeters. The length of the first rigid section is 59 millimeters.

Also, in this example, the collapsible section collapses 28.5 millimeters under a force of 0.03 kg.

### Example 5

The tampon applicator of the present invention can comprise the outer member of the length of 81 millimeters, a tampon positioning member of the length of 51 millimeters, and a tampon of the length of 30 millimeters. The outer member can have a collapsible section and a first rigid section. The length of the collapsible section is 22 millimeters. The length of the first rigid section is 59 millimeters.

Also, in this example, the collapsible section collapses 19.8 millimeters under a force of 0.02 kg.

### Example 6

The tampon applicator of the present invention can comprise the outer member of the length of 81 millimeters, a tampon positioning member of the length of 51 millimeters, and a tampon of the length of 30 millimeters. The outer member can have a first collapsible section, a first rigid section, and a second rigid section. The length of the collapsible section is 22 millimeters. The length of the first rigid section is 20 millimeters. The length of the second rigid section is 39 millimeters.

### Example 7

The tampon applicator of the present invention can comprise the outer member of the length of 89 millimeters, a tampon positioning member of the length of 45 millimeters, and a tampon of the length of 44 millimeters. The outer member can have a first collapsible section, a first rigid section, and a second rigid section. The length of the collapsible section is 30 millimeters. The length of the first rigid section is 18 millimeters. The length of the second rigid section is 41 millimeters.

### Example 8

The tampon applicator of the present invention can comprise the outer member of the length of 90 millimeters, a tampon positioning member of the length of 57 millimeters, and a tampon of the length of 33 millimeters. The outer member can have a first collapsible section, a first rigid section, and a second rigid section. The length of the collapsible section is 23 millimeters. The length of the first rigid section is 23 millimeters. The length of the second rigid section is 43 millimeters.

Also, in this example, the collapsible section collapses 19.5 millimeters under a force of 0.0025 kg.

### Example 9

The tampon applicator of the present invention can comprise the outer member of the length of 102 millimeters, a tampon positioning member of the length of 57 millimeters, and a tampon of the length of 45 millimeters. The outer member can have a first collapsible section, a first rigid section, and a second rigid section. The length of the collapsible section is 30 millimeters. The length of the first rigid section is 30 millimeters. The length of the second rigid section is 42 millimeters.

Also, in this example, the collapsible section collapses 28.5 millimeters under a force of 0.10 kg.

### Example 10

The tampon applicator of the present invention can comprise the outer member of the length of 99 millimeters, a tampon positioning member of the length of 54 millimeters, and a tampon of the length of 44 millimeters. The outer member can have a first collapsible section, a second collapsible section, a first rigid section, and a second rigid section. The length of the first collapsible section is 24 millimeters. The length of the second collapsible section is 20 millimeters. The length of the first rigid section is 18 millimeters. The length of the second rigid section is 57 millimeters.

Also, in this example, the collapsible section collapses 35 millimeters under a force of 0.01 kg.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon applicator for positioning a tampon inside a vaginal cavity, said applicator comprising:
a) an outer member comprising a substantially rigid first section and a collapsible section adjacent to said first section, said tampon being housed within said outer member and
b) a tampon positioning member being disposed within said outer member and co-axially with said tampon for expelling said tampon from said outer member.

2. The tampon applicator according to claim 1 wherein said tampon positioning member is fixedly joined to said outer member.

3. The tampon applicator according to claim 1 wherein said tampon positioning member is slideably joined to said outer member.

4. The tampon applicator according to claim 1 wherein said tampon applicator has a collapsed length and said tampon is at least partially housed within said outer member.

5. The tampon applicator according to claim 1 wherein said tampon positioning member comprises a projection extending radially from within said outer member.

6. The tampon applicator according to claim 1 wherein said tampon positioning member comprises a tubular member.

7. The tampon applicator according to claim 1 wherein said tampon remains in contact with said tampon positioning member at the most proximal position of the tampon applicator.

8. The tampon applicator according to claim 1 wherein said collapsible section comprises plastic material comprising a multiplicity of corrugations.

9. The tampon applicator according to claim 1 wherein said collapsible section collapses at a force of less than about 0.03 kg/force.

10. The tampon applicator according to claim 1 wherein said collapsible section comprises an initial length and wherein said initial length collapses at least about 20% of said initial length.

11. The tampon applicator according to claim 1 further comprising a substantially rigid second section abutting said collapsible section.

12. A tampon applicator for placing a tampon inside a vaginal cavity, said applicator comprising:
a) an outer member comprising a substantially rigid first section, a substantially rigid second section positioned oppositely to said first section, and a collapsible section disposed therebetween, said tampon being partially housed within an outer member and
b) a tampon positioning member being disposed within said outer member and co-axially with said tampon for expelling said tampon from said outer member and for placing said tampon into said proper position within said vaginal cavity.

13. The tampon applicator according to claim 12 wherein said tampon positioning member is slideable within said outer member.

14. The tampon applicator according to claim 12 wherein said outer member comprises a first end and a second end positioned opposite to said first end, said tampon positioning member is positioned between said first end and said second end of said outer member while the tampon applicator has a collapsed length.

15. The tampon applicator according to claim 12 wherein said tampon applicator has a collapsed length and said tampon is at least partially housed within said outer member.
